(19) United States
(12) Patent Application Publication
(10) Pub. No.: US 2019/0218126 A1
(43) Pub. Date: Jul. 18, 2019

(54) FORMATE CATALYSIS FROM HYPERSALINE ENVIRONMENTS BY A HALOTOLERANT HALOMONAS SP.

(71) Applicant: Covestro Deutschland AG, Leverkusen (DE)

(72) Inventors: Heike HECKROTH, Odenthal (DE); Christoph HERWIG, Vienna (DE); Donya KAMRAVAMANESH, Vienna (AT); Paul RUSCHITZKA, Vienna (AT)

(73) Assignee: Covestro Deutschland AG, Leverkusen (DE)

(21) Appl. No.: 16/327,608

(22) PCT Filed: Aug. 24, 2017
(86) PCT No.: PCT/EP2017/071334

§ 371 Date: Feb. 22, 2019

(30) Foreign Application Priority Data
Aug. 25, 2016 (EP) 16185666.1

(51) Int. Cl. C02F 3/34; C25B 1/34; C12N 1/20

(52) U.S. Cl. CPC C02F 3/348; C02F 2101/34; C12N 1/20; C25B 1/34

(57) ABSTRACT

The present invention relates to a method for reducing the formate content of hypersaline wastewater with cells of the Halomonas sp. strain MA-C. The present invention further concerns a method for the production of chlorine and/or sodium hydroxide. Further en-compassed by the present invention is a composition comprising hypersaline wastewater and cells of the Halomonas sp. strain MA-C.

The present invention relates to a method for reducing the formate content of hypersaline wastewater with cells of the Halomonas sp. strain MA-C. The present invention further concerns a method for the production of chlorine and/or sodium hydroxide. Further encompassed by the present invention is a composition comprising hypersaline wastewater and cells of the Halomonas sp. strain MA-C.

The chloralkali process is an industrial process for the electrolysis of NaCl. It is the technology used to produce chlorine and sodium hydroxide. Hypersaline solutions are an important material for the chloralkali process. However, very pure hypersaline solutions are needed. Therefore, it is difficult to use recycled hypersaline wastewater for the chloralkali process. For example, hypersaline wastewater of polyurethane production contains about 300-500 mg/l formate, which has to be removed, otherwise chlorine will be contaminated with CO₂.

The removal of undesired total organic carbon (TOC) can be achieved by adsorption on activated carbon. This is however not possible for formate which does not adsorb to activated carbon.

Formate can be degraded by certain microbial cells, e.g. by formation of enzymes such as formate dehydrogenases. However, microbial cells usually do not grow in hypersaline solutions such as polyurethane salt water which has salt concentration of more than 10%.

Thus, means and methods for reducing the formate content of hypersaline wastewaters are highly required.

WO 2013/124375 discloses the reduction of total organic carbon by certain halophilic and/or haloalkaliphilic microorganisms.

Pendashteh et al. disclose the biological treatment of produced water in a sequencing batch reactor by a consortium of isolated halophilic microorganisms (Pendashteh, Fakhru'l Razi, Chuah, Radiah, Madaeni, and Zurina, Environ. Technol., vol. 31, (2010) pp 1229-1239).

Hinteregger and Streichsbier disclose a moderately halophilic Halomona strain for biotreatment of saline phenolic waste-water (Hinteregger and Streichsbier, Biotechnol. Lett., vol. 19 (1997) pp 1099-1102).

A detailed phenotypic characterization of strains of Halomonas species is provided by Mata Martínez-Cánvas et al. (Mata Martínez-Cánovas, Quesada, and Bëjar, Syst. Appl. Microbiol., vol. 25 (2002) pp 360-375)

Woolard and Irvine disclose the treatment of hypersaline wastewater in the sequencing batch reactor (Woolard and Irvine, Water Res., vol. 29 (1995) pp 1159-1168).

Azachi et al. disclose the isolation of a halotolerant Gram-negative eubacterium from soil collected at a storage site for formaldehyde (Azachi, Henis, Oren, Gurevich, and Sarig, Can. J. Microbiol., vol. 41 (1995): 548-553). The strain, named MA-C and identified as Halomonas sp. (DSM 7328), grew at high salt concentrations. An inducible NAD-dependent formate dehydrogenase activity was detected. Experiments with extracts of the Halomonas sp. strain MA-C showed that the formate dehydrogenase had its temperature optimum at 45° C. The activity of the formate dehydrogenase was strongly inhibited by the presence of salt. At a concentration of 1.5% NaCl, a 50% inhibition was observed.

The technical problem underlying the present invention can be seen as the provision of methods for complying with the aforementioned needs. The technical problem is solved by the embodiments characterized in the claims and herein below.

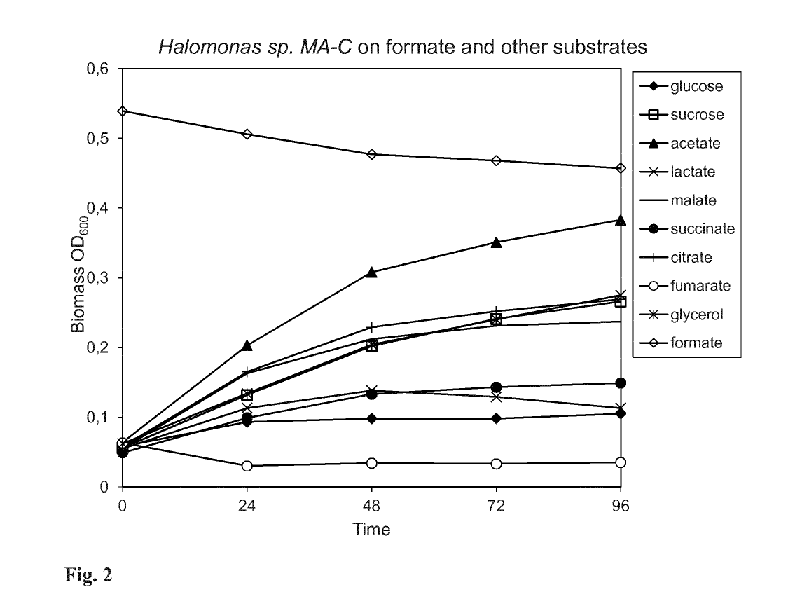

Advantageously, it was shown in the studies of the present invention that the Halomonas sp. strain MA-C allows for an efficient reduction of the formate content of wastewater even in the presence of high concentrations of more than 10% NaCl. Interestingly, the Halomonas sp. strain MA-C did not grow on formate. The removal of formate by the present invention thus takes place via catalytic action of the MA-C cells on formate. The catalytic removal of formate is quick, efficient and the results suggest the metabolite produced by the catalytic reaction is CO₂ as no growth on formate alone was observed (see FIG. 2).

Accordingly, the present invention relates to a method for reducing the formate content of hypersaline wastewater, said method comprising
(i) providing a composition A comprising hypersaline wastewater, and
(ii) mixing said composition A with cells of the Halomonas sp. strain MA-C, thereby generating a composition B comprising the hypersaline wastewater and the Halomonas sp. strain MA-C.

In step (i) of the method of the present invention, a composition A comprising hypersaline wastewater shall be provided. Said composition A is a solution which comprises formate. Preferably, composition A comprises formate in an amount of more than 25 mg/l, in particular in an amount of more than 50 mg/l. More preferably, composition A comprises formate in an amount of more than 50 mg/l but less than 2000 mg/l. Even more preferably, composition A comprises formate in an amount of more than 100 mg/l but less than 1000 mg/l. Most preferably, composition A comprises formate in an amount of more than 100 mg/l but less than 500 mg/l.

In addition to formate, composition A shall comprise NaCl at a high concentration. In a preferred embodiment, composition A comprises NaCl in a concentration of more than 10% (w/v), based on the total volume of composition A. In a further preferred embodiment, composition A comprises NaCl in a concentration of more than 12.5% (w/v), based on the total volume of composition A. Further, it is envisaged that composition A comprises NaCl in a concentration of more than 14% (w/v), based on the total volume of composition A. Moreover, composition A may comprise NaCl in a concentration of more than 15% (w/v), based on the total volume of composition A.

In principle, composition A could comprise NaCl in a concentration up to the saturation concentration of NaCl since has been shown in the studies underlying the present invention that the formate content was reduced even at a NaCl concentration of 20.0% (w/v). Thus, the upper limit for the concentration is, in principle, the saturation concentration of NaCl. However, it is envisaged that composition A comprises NaCl in concentration of less than the saturation concentration. Preferably, the NaCl concentration in composition A is less than 22% (w/v), more preferably less than 20% (w/v) and most preferably less than 18% (w/v), again based on the total volume of composition A. Thus, e.g. the concentration of NaCl may be more than 10% (w/v) but less than 20% (w/v), or more than 12.5%, 14%, or 15% (w/v) but less than 20% (w/v).

The hypersaline wastewater is preferably industrial wastewater, in particular brine. The concentrations of NaCl as referred to above can be found in various industrial wastewaters. In a preferred embodiment, the hypersaline wastewater is derived from methylene diamine production as a preproduct of polyurethanes. Accordingly, step i) of the method of the present invention may comprise the isolation of hypersaline wastewater from methylene diamine production. The hypersaline wastewater might have been subjected to previous steps. In an embodiment, the hypersaline wastewater has been subjected to a purification step with activated charcoal, thereby reducing the TOC content. Further, the wastewater might have been filtered. Further, it is envisaged that the NaCl concentration of the wastewater is concentrated prior to step i) of the method of the present invention. Preferred methods for concentration a composition comprising NaCl are described elsewhere herein.

As set forth above, composition A shall comprise the hypersaline wastewater. In a preferred embodiment, composition A essentially consists of the hypersaline wastewater. In a particular preferred embodiment composition A consists of the hypersaline wastewater. In this case, the terms “hypersaline wastewater” (i.e. the untreated hypersaline wastewater) and “composition A” can be used interchangeably.

The hypersaline wastewater and thus composition A may comprise additional organic compounds such as aniline, phenolate and/or MDA (4, 4′-Methylenedianiline). In an embodiment composition A comprises 0.1 to 20 mg/l, in particular 1 to 10/mg/l aniline and/or 0.1 mg/l to 10 mg/l, in particular 1 to 5 mg/l MDA, and/or 2 to 100 mg/l, in particular 5 to 20 mg/l phenolate.

Preferably, composition A has a total organic carbon (“TOC”) content of more than 50 mg/l, more preferably of more than 60 mg/l, even more preferably of more than 60 mg/l, and most preferably of more than 65 mg/l. Further, it is envisaged that composition A has a TOC (total organic carbon) content of more than 70 mg/l, in particular of more than 70 mg/l. Preferably the composition A can have a total organic carbon (“TOC”) content of up to 1000 mg/l and more.

In accordance with the present invention, the formate content of hypersaline wastewater shall be reduced. The term “reducing” as used herein shall refer to a significant reduction of the formate content of the hypersaline wastewater. Preferably, the term denotes a decrease of the formate content of at least 30%, at least 50%, at least 70% or in particular of at least 90% or of at least 95% of the total formate content present in composition A. Further, it is envisaged that the formate content is eliminated completely.

Preferably, the treated wastewater comprises formate in an amount of less than 15 mg/l, more preferably less of than 10 mg/l and most preferably less than 5 mg/l after the method of the present invention has been carried out.

By carrying out the method of the present invention, the TOC content will be reduced as well (i.e. in addition to the formate content). Preferably, the treated wastewater has a TOC content of less than 40 mg/l, more preferably of less than 30 mg/l and most preferably of less than 20 mg/l (in particular after separation of the cells as described herein elsewhere).

Preferably, the formate content is reduced by the presence of cells the Halomonas sp. strain MA-C, in particular by the activity of a formate dehydrogenase which catalyzes the oxidation of formate to carbon dioxide. The formate dehydrogenase is expressed by the cells of the Halomonas sp. strain MA-C.

According to step ii) of the method of the present invention, composition A as provided in step i) is mixed with cells of the Halomonas sp. strain MA-C (DSM 7328). Thereby, a composition B is generated comprising composition A (and thus the hypersaline wastewater) and the Halomonas sp. strain MA-C (i.e. cells of this strain).

The Halomonas sp. strain MA-C (DSM 7328) has been described in the art. The strain was e.g. described by Azachi et al. (Can. J. Microbiol., vol. 41 (1995): 548-553) and Oren et al. (Biodegradation (1992), 3: 387-398) both of which are herewith incorporated by reference in their entirety. The strain MA-C has been deposited by A. Oren and M. Azachi in the DSM (Deutsche Sammlung von Mikroorganismen and Zellkulturen, Braunschweig, Germany) under DSM number 7328. “MA-C” is the strain designation. The strain is held in the public collection of the DSM and can be ordered from DSM without restriction.

The cells to be mixed with composition A in step ii) shall be viable, i.e. living cells. Accordingly, it is not envisaged to use extracts such as enzyme extracts of the Halomonas strain MA-C. How to assess whether cells are viable, or not, can be assessed by well-known methods. Of course, a certain percentage of the cells to be mixed with composition A might not be viable. However, this is taken into account by the skilled person.

Preferably, a suspension of cells of the strain MA-C is mixed with composition A. The cells are preferably derived from a pre-culture of cells of the MA-C strain. In an embodiment, the preculture of the cells has been carried out in the presence of formate, i.e. the medium for the preculture shall contain formate.

The amount of cells of the strain MA-C to be mixed with composition A can be determined by the skilled person. The cell amount to be mixed shall allow for a sufficient reduction of the formate content. The amount e.g. depends on the volume of composition A to be treated by the method of the present invention. In general, the larger the volume of composition A to be treated, the larger shall be the amount of cells to be used. This will be taken into account by the skilled person.

The mixing may take place in a suitable container. In an embodiment the mixing is carried out in a bioreactor. The term “bioreactor” as used herein refers to a system in which conditions are closely controlled to permit the reduction of the formate content. In an embodiment, said bioreactor is a stirred tank reactor. Preferably, the bioreactor is made of a non-corrosive material such as stainless steel. The bioreactor can be of any size as long as it is useful for the incubation of composition B. Preferably, the bioreactor allows for a large scale reduction of the formate content of hypersaline wastewater. Therefore, it is envisaged that the bioreactor has a volume of at least 1, 10, 100, 500, 1000, 2500, or 5000 liters or any intermediate volume. However, it is also envisaged to carry out the method of the present invention at a low scale, such as with 5 to 100 ml of composition B.

Composition B may further comprise media components which allow for the reduction of the formate content by cells of the strain MA-C. Such media components are well known in the art and include e.g. NH₄Cl, KH₂PO₄, Na₂SO₄, MgCl₂ (e.g. MgCl₂*6H₂O), CaCl₂ (e.g. CaCl₂*2H₂O), and KCl. In an embodiment, composition B comprises a phosphor source, a nitrogen source, a sulfur source, a potassium source and/or a magnesium source (as media components). Composition B may further comprise trace elements such as iron, copper, zinc and cobalt. In an embodiment, the media components are added to composition B after mixing composition A and the cells (as set forth in step ii)).

The selection of suitable media components can be carried out by the skilled person without further ado. Moreover, the skilled person can determine suitable concentrations of media components without further ado.

For example, the following concentration ranges and concentrations for the following media components are considered as suitable. The present invention is however not limited to the media components referred to above and the following concentration ranges.

Concentration in composition B:
NH₄Cl: 0.5 to 3 g/l, e.g. 1.5 g/l
KH₂PO₄: 0.05 to 0.5 g/l, e.g. 0.15 g/l
MgCl₂*6H₂O: 0.5 to 3 g/l, e.g. 1.1 g/l
CaCl₂*2H₂O: 0.1 to 2 g/l, e.g. 0.55 g/l
KCl: 0.5 to 3 g/l, e.g. 1.66 g/l
Na₂SO₄: 0.5 to 1 g/l. e.g. 0.75 g/l

Further preferred concentrations for media components are specified in Table 3 of the Examples section.

In a preferred embodiment of the present invention, the composition B may comprise a suitable substrate, i.e. a substrate which allows for the growth of the strain MA-C. The substrate is preferably added to composition B. In an embodiment, said substrate is an organic acid or a sugar. Preferably, the substrate is selected from acetate, glucose, sucrose, lactate, malate, succinate, citrate and glycerol. In a particular preferred embodiment, the substrate is acetate. It has been shown in the studies underlying the present invention that the presence of acetate in the medium allowed for the best growth of the strain MA-C (as compared to the other tested substrates).

Preferably, composition B comprises a substrate if the incubation is carried out as continuous process. The substrate shall allow for biomass growth in slow rate in order to achieve stability of formate catalysis in continuous mode.

Suitable concentrations or concentration ranges for the substrate can be determined by the skilled person without further ado. The reduction of the formate content and thus the cultivation of the MA-C is preferably done under carbon limitation. Accordingly, it is envisaged that the concentration of the substrate such as acetate allows for biomass growth at a slow rate. Thereby fresh biomass is produced which catalyze the oxidation of formate to carbon dioxide in a continuous way. Preferably, the substrate is added to composition B in an amount that is completely taken up by the cells. Thus, it is envisaged that the TOC content would not be increased by the addition of the substrate.

For example, it is envisaged that the concentration of the substrate, in particular of the substrates mentioned above, in composition B is 0.5 g/l to 10 g/l, in particular 0.5 g/l to 5 g/l. It is to be understood that the concentration of the substrate will decrease during the process if additional substrate is not added continuously.

In an embodiment of the present invention, the further media components and/or the suitable substrate are (is) added to composition B, in particular after mixing composition A and the cells of the strain MA-C. E.g. the further media components and/or the suitable substrate can be at the beginning of the incubation of composition B or during incubation of composition B (e.g. continuously or as pulse).

Of course, the concentration of the substrate will change during incubation, because the substrate will be metabolized by the cells comprised by composition B at a certain rate. Thus, the substrate concentration might not be constant. Nevertheless, additional substrate might be added during incubation in order to compensate for the decrease of the substrate content.

After mixing the cells and composition A, the resulting composition B is incubated in order to allow for the reduction of the formate content by the MA-C cells. Accordingly, the method of the present invention preferably comprises the further step of incubating composition B. Said incubation is carried out under suitable conditions, i.e. under conditions which allow for the reduction of the formate content by the MA-C cells. Preferably, the in incubation is carried out in a bioreactor.

Preferably, the reduction of the formate content (and thus the incubation of composition B) is carried out at temperature of 15° C. to 45° C., more preferably at a temperature of 18° C. to 32° C., more preferably at a temperature of 20° C. to 30° C., and most preferably at a temperature of 25° C. to 30° C., in particular a temperature of 27° C. to 30° C. It is envisaged that the reduction is carried out at a constant temperature. However, it is also contemplated that the temperature might change during the incubation. In a preferred embodiment of the present invention, the temperature of composition B is monitored during incubation.

In a preferred embodiment of the method of the present invention, composition B is agitated (during incubation). Preferably, composition B is agitated in the range of 100 rpm to 700 rpm, more preferably in the range of 100 rpm to 500 rpm, and most preferably in the range of 200 rpm to 400 rpm.

The incubation is carried out under aerobic conditions. Preferably, aerobic conditions are maintained by adding air or purified oxygen to composition B continuously.

Preferably, composition B has a pH value in the range of 5.5 to 8.5, more preferably 6.0 to 8.0, and most preferably in the range of 6.6 to 7.4. Accordingly, the incubation is preferably carried out at such a pH value. In a preferred embodiment, the pH value of composition B is monitored during incubation. It is envisaged that the pH value is kept constant during cultivation. This can be e.g. achieved by adding HCl or NaOH depending on the co-substrate used.

In accordance with the method of the present invention, it is envisaged that the mixing as set forth in step ii) above does not significantly increase the volume of the resulting composition B (as compared to the volume of composition A). Accordingly, the main component of composition B shall be composition A. Thus, the mixing shall not significantly dilute composition A. The dilution factor is preferably lower than 1.2, more preferably lower than 1.1, and most preferably lower than 1.05. Further, it is envisaged that the dilution factor is lower than 1.03 or 1.02. The term “dilution factor” as used herein preferably refers to ratio of the volume of composition B to the volume of composition A.

In other words, composition B comprises (in particular consists of) at least 80%, more preferably at least 90%, and most preferably at least 95% by weight of composition A, based on the total weight of composition B. Further, it is envisaged that composition B at least 97% or 98% by weight of composition A, based on the total weight of composition B.

Since the dilution factor is negligible, it is envisaged that composition B comprises the same, or essentially the same content of formate and NaCl as composition A.

Accordingly, it is envisaged that composition B comprises formate in an amount of more than 25 mg/l, in particular in an amount of more than 50 mg/l. More preferably, composition B comprises formate in an amount of more than 50 mg/l but less than 2000 mg/l. Even more preferably, composition B comprises formate in an amount of more than 100 mg/l but less than 1000 mg/l. Most preferably, composition B comprises formate in an amount of more than 100 mg/l but less than 500 mg/l. It is to be understood that the formate content will decrease during incubation.

Further, it is envisaged that composition B comprises NaCl in a concentration of more than more than 10% (w/v) and formate, based on the total volume of composition B. In a further preferred embodiment, composition B comprises NaCl in a concentration of more than 12.5% (w/v), based on the total volume of composition B. Further, it is envisaged that composition B comprises NaCl in a concentration of more than 14% (w/v), based on the total volume of composition B. Moreover, composition B may comprise NaCl in a concentration of more than 15% (w/v), based on the total volume of composition B. Also preferably, the NaCl concentration in composition B is less than 22% (w/v), more preferably less than 20% (w/v) and most preferably less than 18% (w/v), again based on the total volume of composition B. Thus, e.g. the concentration of NaCl may be more than 10% (w/v) but less than 20% (w/v), or more than 12.5%, 14% or 15% (w/v) but less than 20% (w/v).

As set forth above, composition B in principle may comprise essentially the same content of and NaCl as composition A. Thus, it is envisaged that composition A comprises NaCl in a concentration of more than more than 10% (w/v), based on the total volume of composition A and that composition B comprises NaCl in a concentration of more than more than 10% (w/v), based on the total volume of composition B. Thus, it is envisaged that composition A comprises NaCl in a concentration of more than more than 12.5% (w/v), based on the total volume of composition A and that composition B comprises NaCl in a concentration of more than more than 12.5% (w/v), based on the total volume of composition B. Further, it is envisaged that both composition A and composition B comprise NaCl in a concentration of more than 14% (w/v), based on the total volume of composition A and B, respectively. Moreover, both composition A and B may comprise NaCl in a concentration of more than 15% (w/v), based on the total volume of composition A and B, respectively. Also preferably, the NaCl concentration in composition A and B is less than 22% (w/v), more preferably less than 20% (w/v) and most preferably less than 18% (w/v), again based on the total volume of composition B. Thus, e.g. the concentration of NaCl may be more than 10% (w/v) but less than 20% (w/v), or more than 12.5%, 14% or 15% (w/v) but less than 20% (w/v).

The concentration of biomass, i.e. of cells of the strain MA-C, can be any concentration that allows for the reduction of the formate content. Various biomass ranges were tested (see Example 2). For example, the biomass concentration can be in a range between 0.2 and 10 g/l, in particular in a range between 0.5 to 4.5 g/l. Optimum biomass concentration for 250 mg/l formate is 1.6 g/l. Thus, it is also envisaged that the biomass concentration is in a range between 1.3 to 1.9 g/l.

Moreover, composition B may comprise aniline, phenolate and/or MDA as described herein above for composition A. Further, composition may have a TOC content as described herein above for composition A (at initiation of the incubation).

As set forth above, the method of the present invention is preferably carried out in a large scale. Accordingly, composition B has preferably a volume of at least 1, 10, 100, 500, 1000, 2500, or 5000 liters or any intermediate volume. However, smaller volumes such as volumes of at least 5 ml or 100 ml are envisaged by the present invention as well (e.g. for tests).

The method of the present invention, in particular the incubation as referred to herein, is preferably carried out as a batch, fed-batch or continuous process, in particular as batch, fed-batch or continuous process with cell retention (preferably in a bioreactor). Accordingly, composition B is incubated under batch, fed-batch, or continuous conditions. The term “batch process” preferably refers to a method of incubating cells in which all the components that will ultimately be used for incubating the cells including the substrate and the further media components, composition A as well as the cells themselves, are provided at the initiation of the incubation process. A batch process is preferably stopped at some point and the treated hypersaline wastewater is isolated. The term “fed-batch process” as used herein refers to a process of incubating cells in which additional components such as the additional media components and/or the substrate are provided to the culture at some time after the initiation of the culture process. The A fed-batch culture is preferably stopped at some point and the cells and/or components in the medium are harvested and the treated hypersaline wastewater is isolated.

In a particularly preferred embodiment, the method of the present invention, and thus the incubation as referred to herein, is carried continuous culture with a mixed feed system using a substrate as referred to above (such as acetate).

In a preferred embodiment, the method of the present invention further comprises the step of separating the cells of the Halomonas sp. strain MA-C from the composition B, thereby giving a composition C. The separation of the cells from composition B shall be carried out after the incubation of composition B, i.e. after the reduction of the formate content.

The resulting composition C (which is herein also referred to as the “treated wastewater”) shall be essentially free of cells of the Halomonas sp. strain MA-C. In other words, composition C shall not comprise the cells.

The separation of cells from composition B can be achieved by all cell retention means that are deemed appropriate. For example, the separation of cells can be achieved by centrifugation, filtration, or by decanting. Preferably, the cells are separated from composition B by filtration.

Further, the cells could be immobilized on beads or a solid support, thereby allowing the separation of the cells from composition B.

If a continuous process is carried out, it is contemplated that the separated cells are fed back to the wastewater.

If the method is carried out in a bioreactor, it is envisaged that the bioreactor comprises means for cell retention. Preferably, the bioreactor comprises a membrane suitable for separating the cells from composition B by filtration.

The resulting composition C, i.e. the treated wastewater, preferably comprises formate in an amount of less than 15 mg/l. More preferably, it comprises formate in an amount of less than 10 mg/l and most preferably less than 5 mg/l. The same applies to composition B.

Further, composition C preferably has a TOC content of less than 40 mg/l, more preferably of less than 30 mg/l and most preferably of less than 20 mg/l.

In a preferred embodiment of the present invention, the method further comprises concentrating the composition C, thereby giving a composition C.

This step will increase the NaCl concentration of the treated wastewater, i.e. the NaCl is upconcentrated in the composition. Preferably, the concentrated composition C* comprises NaCl in a concentration of more than 20.0% (w/v), based on the total volume of composition A, in particular in a concentration of more than 22% (w/v). These NaCl concentrations are ideal concentrations when used in the feed stream of the chloralkali process.

In accordance with the present invention, the up-concentration of composition C can be concentration by any method deemed appropriate. Preferred methods are reverse osmosis, ultrafiltration and nanofiltration. In these methods, a positive osmotic pressure to one side of a filtration membrane. Further, the up-concentration can be achieved by evaporization.

As set forth above, composition A and B may comprise NaCl in concentration of more than 20% (w/v). If these concentrations are used, the concentration step, in principle, could be omitted when subjecting the treated wastewater to the chloralkali process.

The definitions and explanations given herein above apply mutatis mutandis to the following subject-matter of the present invention, in particular to the following method of the present invention for the production of chlorine and/or sodium hydroxide, to the composition of the present invention, the bioreactor of the present invention, and the use of the present invention.

The present invention also relates to a method for the production of chlorine and/or sodium hydroxide, comprising the steps of
(a) providing a composition C according to the method of the present invention or a composition C* according to the method of the present invention, and
(b) subjecting the composition according to (a) to a sodium chloride electrolysis, thereby producing chlorine and sodium hydroxide.

The electrolysis of sodium chloride can be carried by methods well known in the art. Preferably, the electrolysis is membrane cell electrolysis of sodium chloride, in particular membrane electrolysis using oxygen consuming electrodes, diaphragm cell electrolysis of sodium chloride or mercury cell electrolysis of sodium chloride.

Step (a) of the aforementioned method, i.e. the provision of a composition C according to the method of the present invention or of a composition C* according to the method of the present invention, preferably comprises the steps of the method for reducing the formate content of a hypersaline solution.

Accordingly, step a) preferably comprises the steps of
(i) providing a composition A comprising hypersaline wastewater, and
(ii) mixing said composition A with cells of the Halomonas sp. strain MA-C, thereby generating a composition B comprising the hypersaline wastewater and the Halomonas sp. strain MA-C, and
(iii) separating the cells of the Halomonas sp. strain MA-C from the composition B, thereby giving a composition C,

If composition C* is used, step a) preferably comprises the further step of concentrating the composition C, thereby giving a composition C*.

Preferably, composition B is incubated after the mixing step (ii). Said incubation shall be carried out under suitable conditions, i.e. under conditions which allow for the reduction of the fot mate content by the MA-C cells. Preferably, the in incubation is carried out in a bioreactor.

The present invention further concerns the composition B as defined herein above in connection of the method of the present invention. Accordingly, the present invention relates to the composition B of hypersaline wastewater and cells of the Halomonas sp. strain MA-C, wherein said composition comprises NaCl in a concentration of more than 10% (w/v) and formate.

Preferred contents of formate and further preferred NaCl concentrations are disclosed in connection of the method of the present invention for reducing the formate content. In addition, the composition may comprise components (such as further media components and/or a suitable substrate, phenol, aniline etc.) as described above.

Further, the present invention relates to a bioreactor comprising at least 1 l of the composition B of the present invention.

Further, the present invention deals with the use of cells of the Halomonas sp. strain MA-C for reducing the formate content of a composition A comprising hypersaline wastewater. Said composition A preferably comprises NaCl in a concentration of more than 10% (w/v), based on the total volume of composition A, and formate.

Finally, the present invention deals with the use of cells of the Halomonas sp. strain MA-C for reducing the formate content of a composition B.

In accordance with the aforementioned uses, the formate content is preferably reduced as described herein above in connection with the method of the present invention. The definitions and explanations apply accordingly.

### PREFERRED EMBODIMENTS OF THE PRESENT INVENTION

In the following, preferred embodiments of the present invention are described. The definitions and explanations apply mutatis mutandis.
1. A method for reducing the formate content of hypersaline wastewater, said method comprising
   (i) providing a composition A comprising hypersaline wastewater, and
   (ii) mixing said composition A with cells of the Halomonas sp. strain MA-C, thereby generating a composition B comprising the hypersaline wastewater and the Halomonas sp. strain MA-C,
   wherein composition A comprises NaCl in a concentration of more than 10% (w/v), based on the total volume of composition A.
2. The method of embodiment 1, wherein composition A comprises NaCl in a concentration of more than 12.5% (w/v), based on the total volume of composition A.
3. The method of embodiment 1, wherein composition A comprises NaCl in a concentration of more than 15% (w/v), based on the total volume of composition A.
4. The method of any one of embodiments 1 to 3, wherein composition A consists of hypersaline wastewater.
5. The method of any one of embodiments 1 to 4, wherein composition A has a TOC content of more than 50 mg/l.
6. The method of any one of embodiments 1 to 5, wherein composition A comprises formate in an amount of more than 50 mg/l.
7. The method of any of embodiments 1 to 6, wherein composition B has a pH value in the range of 6.0 to 8.0, preferably in the range of 6.6 to 7.4.
8. The method of any one of embodiments 1 to 7, wherein the reduction of the formate content is carried out at temperature of 15° C. to 45° C., preferably at a temperature of 18° C. to 32° C.
9. The method of any one of embodiments 1 to 8, wherein the method is carried out as batch, fed-batch or continuous process in a bioreactor, in particular will cell retention.
10. The method of any one of embodiments 1 to 9, wherein step (i) comprises isolating the hypersaline wastewater from methylene diamine production.
11. The method of any one of embodiments 1 to 10, wherein the composition B comprises NaCl in a concentration of more than 10% (w/v).
12. The method of any one of embodiments 1 to 10, wherein the composition B comprises NaCl in a concentration of more than 15% (w/v).
13. The method of any one of embodiments 1 to 12, wherein composition B further comprises at least one substrate selected from the group consisting of acetate, glucose, sucrose, lactate, malate, succinate, citrate, and glycerol, which substrate is added to composition B.
14. The method of any one of embodiments 1 to 13, wherein composition B has a volume of at least 1 l.
15. The method of any one of embodiments 1 to 14, further comprising separating the cells of the Halomonas sp. strain MA-C from the composition B, thereby giving a composition C.
16. The method of any one of embodiment 15, wherein composition C has a TOC content of less than 30 mg/l.
17. The method of embodiments 15 and 16, wherein composition C comprises formate in an amount of less than 15 mg/l.
18. The method of any one of embodiments 15 to 17, further comprising concentrating the composition C, thereby giving a composition C*.
19. A method for the production of chlorine and sodium hydroxide, comprising the steps of
   (a) providing a composition C according to the method of any one of embodiments 15 to 17 or a composition C* according to the method of embodiment 18, and
   (b) subjecting the composition according to (a) to a sodium chloride electrolysis, thereby producing chlorine and/or sodium hydroxide.
20. A composition B of hypersaline wastewater and cells of the Halomonas sp. strain MA-C, wherein said composition comprises NaCl in a concentration of more than 10% (w/v) and formate.
21. A bioreactor comprising at least 1 l of the composition of embodiment 20.
22. Use of cells of the Halomonas sp. strain MA-C for reducing the formate content of a composition A comprising hypersaline wastewater, wherein said composition comprises NaCl in a concentration of more than 10% (w/v), based on the total volume of composition A, and formate.

All references cited in this specification are herewith incorporated by reference with respect to their entire disclosure content and the disclosure content specifically mentioned in this specification.

In the Figures:

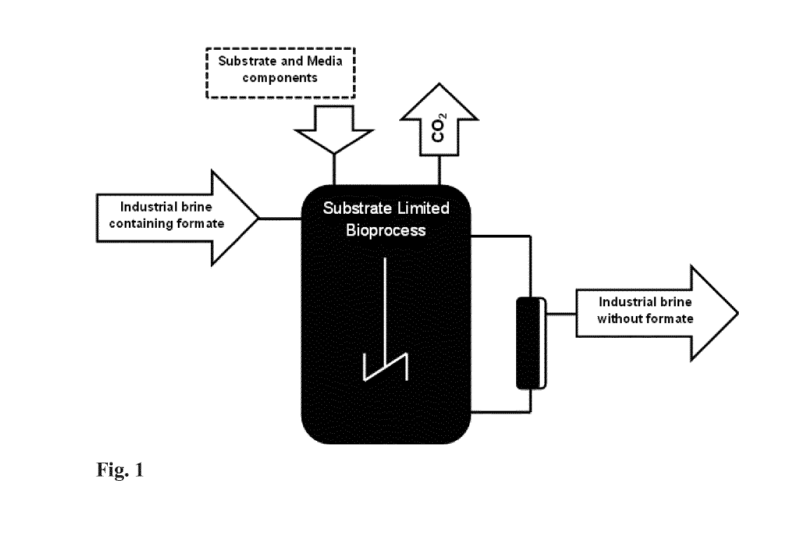

FIG. 1. Represents biological treatment of waste water containing formate in a continuous mode using catalytic activity of MA-C cells and a mixed feed system.

FIG. 2. Shows increase in biomass of Halomonas sp. MA-C cells on various sugars and organic acids. Graph shows no biomass increase on formate as substrate.

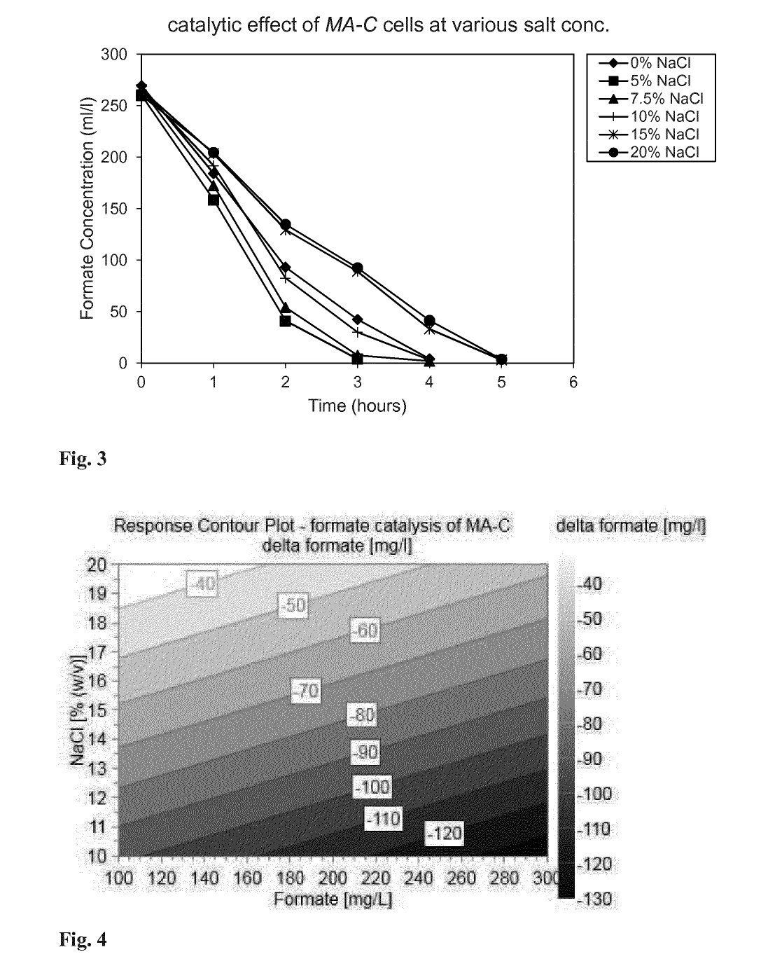

FIG. 3. Shows catalytic activity of Halomonas sp. MA-C cells on 250 mg/l formate in various salt concentrations. Although quicker formate catalysis occurs at lower salt concentrations (0 to 7% w/v NaCl), efficient formate catalysis was also observed at higher salt concentrations (15 to 20% w/v NaCl).

FIG. 4. The response counter plot shows the optimal formate catalysis occurs at lower NaCl concentrations and higher initial formate concentrations.

FIG. 5. Represents effect of biomass concentration of MA-C cells on formate catalysis. Optimum biomass concentration for 250 mg/l formate is 1.6 g/l.

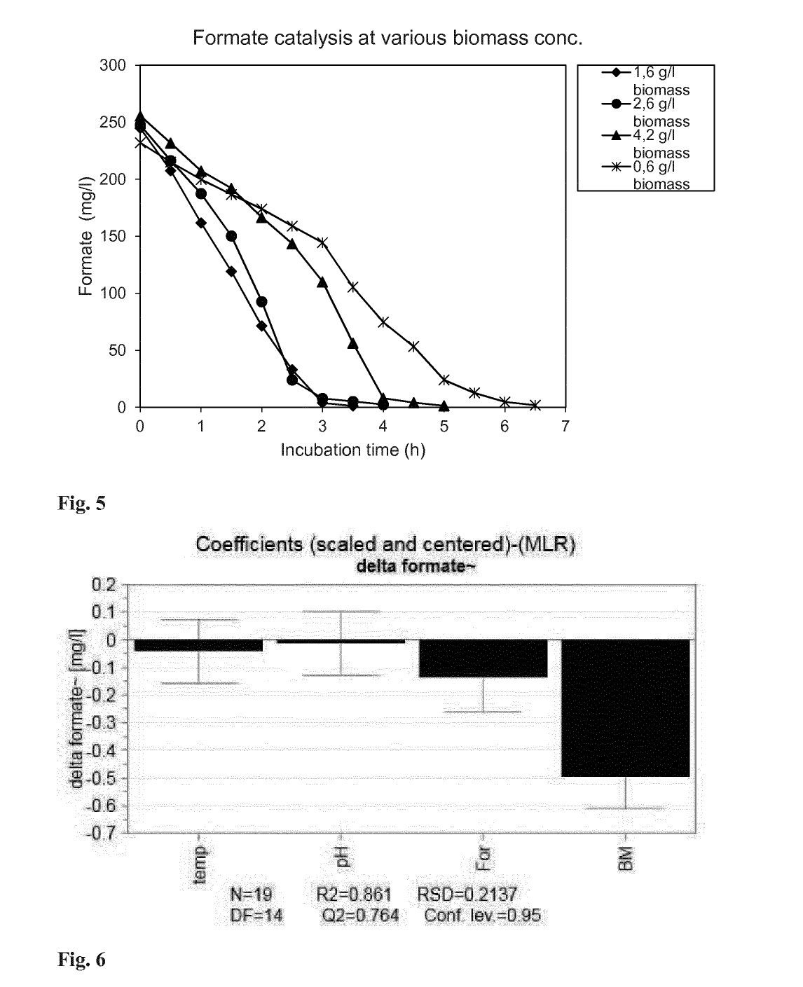

FIG. 6. The coefficient plot shows significance of two factors initial formate concentration and biomass concentration on formate catalysis.

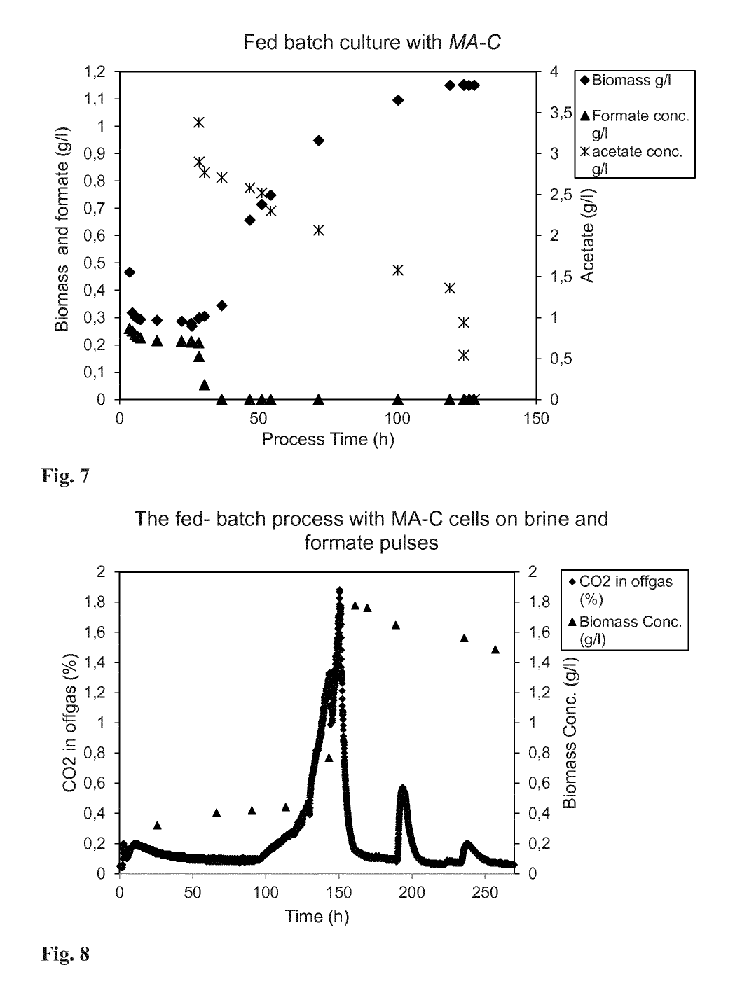

FIG. 7. Shows the fed-batch culture with MA-C cells on the brine containing 14% w/v NaCl and 250 mg/l formate. Biomass is reduced until 28 hour of the process, formate is slightly catalyzed. A pulse of acetate (3.5 g/l) facilitates biomass growth at 28 hour of the process and immediate catalysis of formate occurs. Acetate is completely utilized at 120 hour of the process.

FIG. 8. Represents a batch process with MA-C cells on the brine containing 14% w/v NaCl, 250 mg/l formate and 3.5 g/l acetate. The biomass increase is obtained from acetate. Addition of two formate pulses at 190 and 235 hour of the process does not lead to biomass growth. The CO₂ percentage in offgas shows the biomass in the reactor can catalyze the first pulse of formate completely into CO₂. The second pulse of formate is not catalyzed completely.

The invention will be merely illustrated by the following Examples. The said Examples shall, whatsoever, not be construed in a manner limiting the scope of the invention.

### EXAMPLES

### Example 1: Formate Catalysis in Shake Flask Experiments

### Strain and Media

Halomonas sp. (DSM 7328) designated as MA-C, wild type strain was purchased from dsmz-German collection of microorganisms and cell cultures. Shake-flask cultures for inoculum preparation were grown under 120 rpm and 30° C. in laboratory incubator (Infors, Switzerland) with media no. 1428 suggested by dsmz with following compositions (g/l): NaCl 100, MgCl₂.6H₂O 10, KCl 1.0, Na₂SO₄ 0.5, yeast extract 5.0, Tryptone 5.0; pH 7.0. The 500 ml Erlenmeyer flasks and the media were always sterilized.

### Analytics

Turbidity as indicator for cell growth was measured using Shimadzu UV/Vis spectrophotometer at 600 nm in 12 hour intervals. Residual formate concentration in the culture supernatant was measured using HPLC. The HPLC (Thermo-Fisher) method was performed with an Aminex HPX-87H column from Bio-Rad at 30° C., an isocratic eluent of 0.1% TFA in MQ water with a flow of 0.5 ml/min followed by UV detection at 210 nm. The limit of quantification with injection volume of 20 μl was 5 mg/l for formate. The standards used for quantification were prepared in the same salty matrix as the samples.

### Formate Studies in Shake Flask

For the formate uptake studies a synthetically defined media was prepared. The media composition in g/l was as follows: NaCl 100, MgCl₂.6H₂O 10, KCl 1.0, and Na₂SO₄ 0.5, different concentrations of sodium formate (50 to 2000 mg/l) was added. The pH was adjusted to 7.0. Cells growing on complex media containing: Yeast extract 5.0, Tryptone 5.0, NaCl 100, MgCl₂.6H₂O 10, KCl 1.0, and Na₂SO₄ 0.5 were harvested by centrifugation at 3000 rpm, for 5 minutes. Cells were washed and dissolved in shake-flasks containing 100 ml defined media with only formate as Carbon source and were incubated at 30° C. and 120 rpm agitation. The zero hour OD₆₀₀ was measured and one ml sample was stored for HPLC analysis at zero hour. Growth on formate and the residual formate concentration were monitored.

The cells of Halomonas sp. MA-C fail to use formate as source for growth however the HPLC analysis to determine the residual formate concentration over time show formate was completely removed from the media cultured both on synthetic media and on actual brine. The catalytic action of cells on formate was investigated in more details in more experiments in shake flasks as well as bioreactor in order to be able to control other process parameters.

### Example 2: Halomonas sp. MA-Con More Substrates

The strain was studied for growth on various sugars and organic acids as substrate. Sterile media containing either of the following: glucose, sucrose, acetate, lactate, citrate, malate, succinate, fumarate and the sugar alcohol glycerol in 25 mM concentration was prepared. 100 ml sterile media was transferred into 500 ml Erlenmeyer flasks and uniform concentration of cells was added to each flask and flasks were further incubated at 30° C. with 120 rpm agitation.

Halomonas sp. MA-C can utilize all sugars and organic acids used in this study as source for growth (FIG. 2). No growth was observed on fumarate and the fumarate concentration in the culture supernatant stayed intact. Here we concluded the catalytic activity of Halomonas sp. MA-C is specific to formate.

### Example 3: Optimum Culture Conditions for Formate Catalysis

Catalytic activity of MA-C cells on formate was studied in various salt concentrations and it was observed that efficient formate catalysis occurs at all NaCl concentrations (0 to 20) % w/v, although better formate catalysis occurs at lower salt concentrations (FIG. 3) and (FIG. 4).

In order to find the optimum conditions and influence of other process parameters on catalytic activity of MA-C cells on formate a fractional factorial design of experiment was carried out to evaluate the influence of four factors (Temperature, pH, Formate concentration and biomass concentration) on three parameters (delta biomass concentration, residual formate concentration and pH) The factors studied in this experiment along with respective ranges are given in (Table 1).

| Factor name | Ranges |
|---|---|
| Temperature | 25° to 45° C. |
| pH | 6 to 8 |
| Formate concentration | 100 to 1000 mg/l |
| Biomass concentration | 0.5 to 4.5 g/l |

Nineteen experiments were suggested by the statistical tool, Modde for this study. The experiments were performed in shake flasks on actual brine containing 14% w/v NaCl. Biomass concentration, pH changes and the residual formate concentration were determined at 24 hour intervals. The measurements obtained were analyzed by Modde.

A valid model was obtained for delta formate. The coefficient plots showed influence of initial biomass concentration on formate catalysis (FIG. 5). Better formate removal could be obtained at higher biomass concentrations. Temperature seem to not have an influence on formate catalysis however for cell survival lower temperatures are suggested. pH has no significance on formate catalysis where range of (6 to 8) was studied (FIG. 6).

### Example 4: Cultivation in the Bioreactor

Due to limitations of shake flask experiments further investigations were done in the reactor where process parameters and culture conditions are controlled. The experiments in this case were carried out in special corrosion resistant bioreactor equipment suitable for cultivation at hypersaline environments.

The special non-corrosive Laborfors PEEK (Infors, AG, Switzerlands) reactor was utilized with the following specifications:
Borosilicate glass culture vessel: 1 L volume
Borosilicate glass exhaust gas cooling
Special corrosion resistant Polymer (PEEK) bioreactor top lid
Special corrosion resistant Polymer (PEEK) thermometer holder
Borosilicate glass sampling tube and gas inlet tube
Special corrosion resistant agitator
Borosilicate glass jacket on the reactor vessel

Online Analytics of:
Exhaust gas CO₂
Exhaust gas O₂
Glass pH probe
Hastelloy Clark pO₂ and
Thermal Mass flow controller for air

A batch process was performed in order to investigate the feasibility of cultivation of the Halomonas sp. MA-C in the bioreactor on an actual brine containing 14% w/v NaCl and 250 mg/l of formate. The following media components were further added to the brine: KCl 1 g/l, MgCl₂.6H₂O 10 g/l, Na₂SO₄ 0.5 g/l and Trace elements solution 2.5 ml containing (Fe, Cu, Mn, and Zn).
Temperature: 30° C.
pH: 7.0 (0.5 M HCl was used for pH control)
Agitation: 300 rpm
Air inlet: 0.2 vvm (NL/min)

The reactor was inoculated to a biomass concentration of approximately 0.5 g/l at 3.5 hour of the process. The OD₆₀₀ and residual formate concentration were measured at various time intervals. Biomass concentration showed reduction and formate was catalyzed to a certain extend (FIG. 7) which again confirmed the catalytic action of MA-C cells on formate. The product of formate catalysis was CO₂ which was measured by offgas analyzers. As MA-C cells could only catalyze formate and in this case the substrate no biomass was formed a second substrate was fed in order to produce biomass. The previous experiments showed growth on acetate can take place at highest rates in compare to other simple substrates acetate as a cheap substrate is a suitable alternative in industrial processes. To that end acetate was chosen as the second substrate in the process. As formate reduction was slow a pulse of acetate (3 g/l) was fed to the reactor at 28^{th} hour process time. The biomass started to increase and the fresh biomass formed catalyzed formate completely at 36.7 hour. After 120 hours of the process complete removal of acetate could also be observed (FIG. 7). In order to accelerate acetate uptake and formate degradation in continuous mode media optimization was performed.

### Example 5: Media Optimization for Optimal Formate Catalysis in Continuous Mode

For media optimization purpose, a full factorial design of experiment was carried out to evaluate the influence of five factors (magnesium, potassium, sulfur, nitrogen and phosphorous) on three parameters (biomass concentration, delta formate and delta acetate). All experiments were carried out with 250 mg/l formate and 3.5 g/l acetate and equal concentration of MA-C cells as inoculum in shake flasks. Nineteen experiments were suggested by statistical tool, Modde for the fractional factorial design and two control experiments were also added to the matrix. The biomass and residual formate and acetate concentration were measured every 24 hours. The results obtained after 72 hours were analyzed for the responses. The coefficient plot showed significant effect of nitrogen and potassium on the final biomass concentration compared to the other factors. Also, data obtained from five experiments showed to have the highest final biomass concentrations and complete acetate and formate uptake. The components and the ranges used in this experiments are shown in (Table 2).

| Factor name | Element | Ranges (g/l) |
|---|---|---|
| Magnesium | MgCl₂•6H₂O | 0 to 10 |
| Potassium | KCl | 0 to 1 |
| Sulfur | Na₂SO₄ | 0 to 0.5 |
| Nitrogen | NH₄Cl | 0 to 1.5 |
| Phosphorous | NaH₂PO₄ | 0 to 0.15 |

The experiments with highest biomass concentration and complete uptake of acetate and formate had the following concentrations (g/l) of the media components (Table 3).

| Exp. No | NaH₂PO₄ | NH₄Cl | Na₂SO₄ | KCl | MgCl₂•6H₂O |
|---|---|---|---|---|---|
| N16, N21 | 1 | 1.5 | 0.5 | 1 | 10 |
| N17, 18, 19 | 0.5 | 0.75 | 0.25 | 0.5 | 5 |

### Example 6: Formate Catalysis by Halomonas sp. MA-C Cells in Continuous Mode

After the strain Halomonas sp. MA-C could successfully be cultivated in the bioreactor and formate was fully catalyzed using acetate as the co-substrate a continuously limited process was established using the mixed feed system. The first task was to specify the options for the optimal dilution rate for MA-C which is high enough which could be applicable for industrial scale use still lower than the maximum specific growth rate. The following dilution rates were experimented:
0.008 l/h
0.011 l/h
0.015 l/h
0.02 l/h the wash out occurred which means, this dilution rate is bigger than the maximal specific growth rate of the cells on acetate. In fact the maximum growth rate was determined from the batch experiments to be close to 0.02 l/h. In order to apply higher dilution rates and avoid the wash out use of cell retention seems necessary.

Prior to the experiment all the media components along with 3.5 g/l acetate were added to the brine. pH was kept at 7.0 constantly by addition of 0.5 M HCl. In order to obtain steady states from the continuous processes samples were obtained from the process in order to measure biomass concentration. Residual concentration of formate and acetate were measured by HPLC show complete removal of acetate and formate at steady state. Analysis of TOC content of treated brine were done externally and the TOC content showed significant reductions to about 15 ppm. Rates and the yields on the substrates (oxygen: Yo₂/s, carbon dioxide: Yco₂/s Biomass: Y x/s) for both acetate and formate were calculated. Overall carbon balances were calculated as 0.918 Cmol/Cmol.

1.-15. (canceled)

16. A method for reducing the formate content of hypersaline wastewater, said method comprising
(i) providing a composition A comprising hypersaline wastewater and formate, and
(ii) mixing said composition A with cells of the Halomonas sp. strain MA-C, thereby generating a composition B comprising the hypersaline wastewater and the Halomonas sp. strain MA-C,
wherein composition A comprises NaCl in a concentration of more than 10% (w/v), based on the total volume of composition A.

17. The method of claim 16, wherein composition A comprises NaCl in a concentration of more than 12.5% (w/v), based on the total volume of composition A, or wherein composition A comprises NaCl in a concentration of more than 15% (w/v), based on the total volume of composition A.

18. The method of claim 16, wherein composition A consists of hypersaline wastewater.

19. The method of claim 16, wherein composition A comprises formate in an amount of more than 50 mg/l.

20. The method of claim 16, wherein the reduction of the formate content is carried out at temperature of 15° C. to 45° C.

21. The method of claim 16, wherein step (i) comprises isolating the hypersaline wastewater from methylene diamine production.

22. The method of claim 16, wherein the composition B comprises NaCl in a concentration of more than 10% (w/v), or wherein the composition B comprises NaCl in a concentration of more than 15% (w/v).

23. The method of claim 16, wherein composition B further comprises at least one substrate selected from the group consisting of acetate, glucose, sucrose, lactate, malate, succinate, citrate, and glycerol, which substrate is added to composition B.

24. The method of claim 16, further comprising separating the cells of the Halomonas sp. strain MA-C from the composition B, thereby giving a composition C.

25. The method of any one of claim 24, wherein composition C has a TOC content of less than 30 mg/l, and/or wherein composition C comprises formate in an amount of less than 15 mg/l.

26. The method of claim 24, further comprising concentrating the composition C, thereby giving a composition C*.

27. A method for the production of chlorine and/or sodium hydroxide, comprising the steps of
(a) providing a composition C according to the method of claim 24 or a composition C* according to the method of claim 26, and
(b) subjecting the composition according to (a) to a sodium chloride electrolysis process, thereby producing chlorine and sodium hydroxide.

28. The method of claim 27, wherein the sodium chloride electrolysis is selected from membrane cell electrolysis of sodium chloride, in particular membrane electrolysis using oxygen consuming electrodes, diaphragm cell electrolysis of sodium chloride and mercury cell electrolysis of sodium chloride.

29. A composition B of hypersaline wastewater and cells of the Halomonas sp. strain MA-C, wherein said composition comprises NaCl in a concentration of more than 10% (w/v) and formate and/or a bioreactor comprising at least 1 l of said composition.

30. A method comprising utilizing cells of the Halomonas sp. strain MA-C for reducing the formate content of a composition A comprising hypersaline wastewater, wherein said composition comprises NaCl in a concentration of more than 10% (w/v), based on the total volume of composition A, and formate.
